(19) United States
(12) Patent Application Publication
(10) Pub. No.: US 2007/0276449 A1
(43) Pub. Date: Nov. 29, 2007

(54) Interactive transcutaneous electrical nerve stimulation device

(75) Inventors: M. Lee Gunter, League City, TX (US); Michael Distler, Kemalt, TX (US); Viktoriya V. Gunter, League City, TX (US)

Correspondence Address: Hugh R. Kress;ARNOLD & FERRERA, L.L.P., 2401 Fountainview, Suite 630, Houston, TX, 77057, US

(73) Assignee: Med-Lectric Corporation

(21) Appl. No.: 11/153,814

(22) Filed: Jun. 15, 2005

(51) Int. Cl. A61N 1/04

(52) U.S. Cl. 607/46

(57) ABSTRACT

A wireless, handheld electrical therapy device delivers electrical pulses to a treatment area of a patient. In one embodiment, the device comprises a microcontroller-based pulse generator circuit selectively operable in a plurality of therapeutic modes. The device comprises an ergonomic housing adapted to be comfortably grasped by a user. A plurality of electrodes are disposed on a surface of the housing. In operation, a user brings the electrodes into contact with a patient's skin at a location on the patient to be treated. Electrical pulses are delivered between the electrodes, thereby electrically stimulating neural tissue at the treatment location. In one embodiment, the device is operable in a manual mode wherein the user selects from among a plurality of therapeutic regimens each corresponding to a set of predetermined operational parameters. Among the variable operational parameters are pulse amplitude, frequency, duration, damping, and shape. In another embodiment of the invention, the device is operable in an automatic mode wherein electrical conditions at the skin surface are periodically sensed and the operational parameters automatically adjusted to achieve optimal therapeutic effectiveness.

### FIELD OF THE INVENTION

This invention relates generally to the field of therapeutic devices, and more particularly relates to an interactive device for electrical stimulation of body tissue.

### BACKGROUND OF THE INVENTION

Transcutaneous electrical nerve stimulation (TENS) has been an accepted mode of physical therapy for many years, and is well characterized in the literature. U.S. Pat. No. 4,147,171 to Greene et al., entitled “Transcutaneous Pain Control and/or Muscle Stimulating Apparatus,” is representative of relatively early electrotherapeutic devices. On the other hand, U.S. Pat. No. 6,445,955 to Michelson et al., entitled “Miniature Wireless Transcutaneous Electrical Neuro or Muscular Stimulation Unit,” represents a more state-of-the-art implementation of a TENS unit.

TENS has primarily been understood as being intended to provide pain relief via a nerve signal blocking mechanism. TENS devices typically deliver monophasic or biphasic electrical stimulating pulses between 10 milliamperes (mA) and 100 mA in amplitude. Pulse amplitude, pulse width, and pulse rate are often user-adjustable. The stimulus pulse is typically delivered between a pair of electrodes that are manually disposed over major muscle groups or nerves that are to receive the stimulation. A variety of TENS devices are commercially available for clinical and public use.

Microcurrent electrotherapy, or microcurrent electrical neuromuscular stimulation (MENS) is gaining popularity in clinical practice for decreasing or eliminating pain and stimulating healing processes. MENS is typically used for pain relief and more typically for tissue healing by affecting the injured tissue at a cellular level. However, the exact mechanisms by which microcurrent therapy works have yet to be completely understood.

Present day electrotherapy units have a number of limitations which affect their functionality as an electrotherapy tool. First, there are a number of problems with attaching external electrodes. Electrodes must adhere to the skin either with an adhesive or tape. Over time, the adhesive or tape becomes loose, rendering the therapy ineffective. This holds true particularly with active patients, even those doing light exercise or normal daily activities. Second, the placement of external electrodes must be done properly. The average patient has a poor understanding of anatomical features, leading to underutilization of the electrodes or, worse yet, improperly placed electrodes, potentially leading to unnecessary or improper treatment.

Third, the wires and electrodes are challenging to place in or through clothing, so as to be inconspicuous despite the relatively small size of the device delivering the electrotherapy. Problems with prior art electrotherapy devices include, without limitation, detachment of lead wires from the electrodes or stimulator during patient movements, interference of lead wires with daily activities, and bulkiness that leads to decreased use of the stimulator unit.

Certain of the known deficiencies in prior art electrotherapeutic devices are acknowledged in the above-referenced Michelson et al. '955 patent. For example, the '955 patent emphasizes the small size of the disclosed device, facilitating its insertion into a splint, bandage, brace or cast. However, the device disclosed in the '955 patent does not appear to fully address the issues of proper electrode placement and adhesion of the electrodes to the patient's skin. Further, although the '955 patent characterizes the device disclosed therein as “wireless,” this is believed to be a misleading characterization. In each embodiment disclosed in the '955 patent, the electrodes are carried on an electrode assembly separate from the stimulator device itself. That is, the '955 patent at best merely substitutes conductive tapes or elastomers for “wires,” in establishing a connection between the pulse generator and the separate electrodes or electrode assemblies.

Accordingly, there remains an ongoing need for a different electrotherapy delivery mechanism and portable electrotherapy device capable of delivering multiple modes of operations to an injured site and a variety of injury-related conditions.

### SUMMARY OF THE INVENTION

In view of the foregoing, the present invention is directed to a hand-held electrotherapy device which can be used by either hand of a clinician or patient. Further, the device is capable of delivering a variety of waveforms to patient tissue, with varying amplitude according to the patient's comfort level, so as to be capable of treating a variety of physical conditions both acute and chronic.

In one embodiment, the invention comprises a hand-held unit having three built-in (integral) electrodes thereon. The electrodes are located opposite the control interface, which faces the operator. An internal electronics unit contains an electronic control circuit and at least one power source (battery) to provide operational power to the device. An LED panel containing an array of indicators, is provided on a rear surface of the housing, allowing the operator to verify the modes and intensity of operation.

The control circuit regulates operation of the electronics through a plurality of different modes, each being intended to administer treatment via a specific waveform. In one embodiment, eight levels of intensity are available.

The operating keys are arranged in such a way that one can easily and quickly change any setting suitable to one's tolerance to the stimulating pulses. In accordance with one aspect of the invention, and unlike prior art electrotherapy devices, the electrodes need not be placed directly on the injured site, but rather only generally in the area of treatment. Due to the nature of the waveforms generated by the device, a treatment site receives a wide range of treatment and effectively treats an area greater than the precise area of immediate contact with the electrodes.

In accordance with another aspect of the invention, aside from the ease and facility of use compared with prior art devices, the present invention emits a unique sound when placed on the skin. On an injured site, there is little or no sound emitted. As treatment progresses, the sound increases in intensity, such that along with the LED display, the device indicates both visually and aurally the progression of treatment.

### BRIEF DESCRIPTION OF THE DRAWINGS

The foregoing and other features and aspects of the present invention will be best understood with reference to the following detailed description of a specific embodiment of the invention, when read in conjunction with the accompanying drawings, wherein:

FIGS. 1a, 1b, and 1c are rear, side, and front views, respectively, of a wireless, handheld electrotherapy device in accordance with one embodiment of the invention;

FIG. 2 is a functional block diagram of the operational circuitry in the device of FIGS. 1a, 1b, and 1c;

FIG. 3 is a schematic diagram of the operational circuitry in the device from FIG. 2;

FIG. 4 is a plot of a voltage waveform generated by the device from FIG. 2;

FIG. 5 is a plot of a voltage waveform of a therapy regimen delivered by the device from FIG. 2;

FIG. 6 is a plot of a voltage waveform of another therapy regimen delivered by the device from FIG. 2; and

FIG. 7 is a plot of a voltage waveform of another therapy regimen delivered by the device from FIG. 2.

### DETAILED DESCRIPTION OF A SPECIFIC EMBODIMENT OF THE INVENTION

In the disclosure that follows, in the interest of clarity, not all features of actual implementations are described. It will of course be appreciated that in the development of any such actual implementation, as in any such project, numerous engineering and technical decisions must be made to achieve the developers' specific goals and subgoals (e.g., compliance with system and technical constraints), which will vary from one implementation to another. Moreover, attention will necessarily be paid to proper engineering and programming practices for the environment in question. It will be appreciated that such a development effort might be complex and time-consuming, but would nevertheless be a routine undertaking for those of ordinary skill in the relevant fields.

Furthermore, for the purposes of the present disclosure, the terms “comprise” and “comprising” shall be interpreted in an inclusive, non-limiting sense, recognizing that an element or method step said to “comprise” one or more specific components may include additional components.

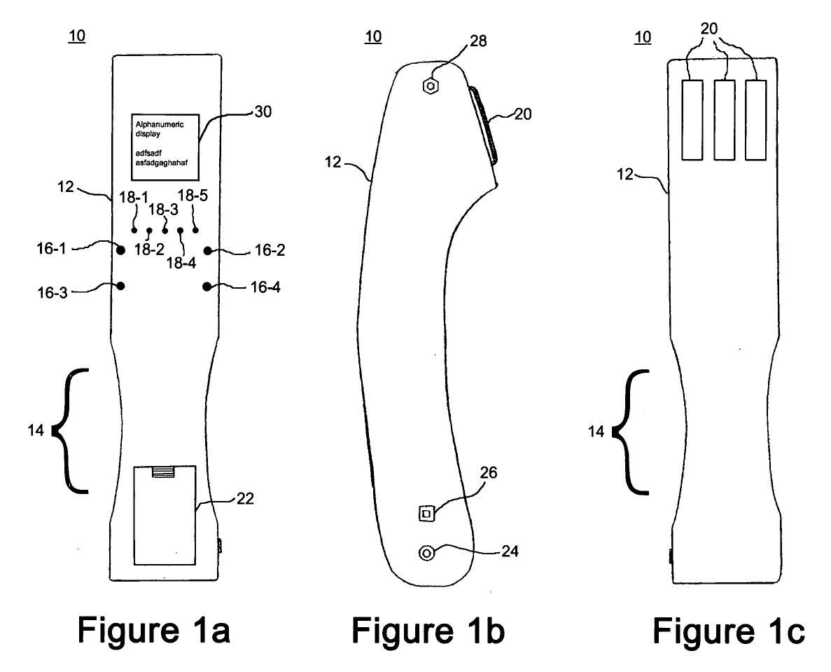

Referring to FIGS. 1a, 1b, and 1c, there are shown rear, side, and front views, respectively, of a handheld, wireless electrotherapy device 10 in accordance with one embodiment of the invention. As shown in FIGS. 1a, 1b, and 1c, device 10 comprises an external housing 12 having a symmetrical, ergonomic design such that it can be easily grasped with one hand at a base portion thereof designated generally with reference numeral 14.

With reference specifically to FIG. 1a, a plurality of control buttons 16-1, 16-2, 16-3, and 16-4 (collectively, control buttons 16) are disposed on the back of housing 12. The respective functions of these buttons are as follows:

| Button | Function |
|---|---|
| 16-1 | on/off |
| 16-2 | power increase |
| 16-3 | mode select |
| 16-4 | activate treatment |

Notably, buttons 16 are positioned on the back of housing 12 in such a way as to be readily actuated by the index finger of an operator grasping device 10 at lower region 14 as previously described.

Positioned above buttons 16-1 through 16-4 is an array of LED indicators 18-1 through 18-5 (collectively, indicators 18). The functions of these respective indicators are as follows:

| Indicator | Function |
|---|---|
| 18-1 | Auto mode indicator (on/off) |
| 18-2 | Amplitude modulation indicator (on/off) |
| 18-3 | Frequency modulation indicator (on/off) |
| 18-4 | Dosage level achievement indicator (on/off) |
| 18-5 | Output intensity indicator (variable intensity) |

Referring to FIGS. 1b and 1c, disposed generally on a distal face portion of the surface of housing 12 are a plurality of electrodes 20. In the preferred embodiment, three electrodes 20 are provided, as will be hereinafter discussed in further detail.

A battery compartment 22 is shown in FIG. 1a. A jack 24 is provided for an external battery charger (not shown) to be connected to device 10 to recharge the battery.

With reference to the side view of FIG. 1b, in one embodiment, a jack 26 for interfacing device 10 with a computer or other external programming/control system (also not shown) is provided. For example, jack 26 may be a USB port for interfacing device 10 with a personal computer or the like.

Likewise, a jack 28 may be provided for permitting device to be used with an external electrode assembly. While not the preferred method of using device 10, there are some instances where the use of an external electrode to apply electrotherapeutic pulses may be necessary, such as treatment to areas of a patient's face, for example.

Finally, as shown in FIG. 1a, in one embodiment of the invention, an optional alphanumeric display 30 may be provided for displaying operational data, instructions for use, device settings, and other information.

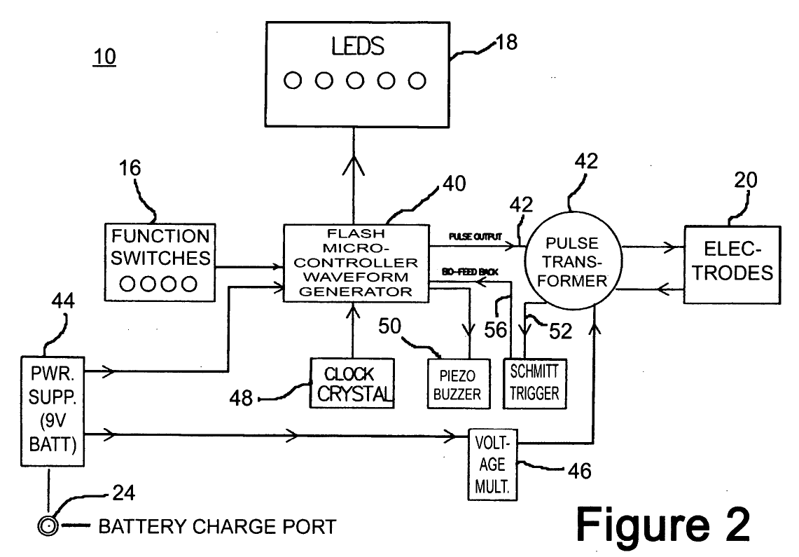

Turning to FIG. 2, there is shown a simplified block diagram of the electrical components of device 10 in accordance with one embodiment of the invention. As shown in FIG. 2, device 10 comprises a microcontroller 40, which among other functions serves as a waveform generator for generating electrotherapy pulses delivered to patient tissue via electrodes 20 carried on the surface of housing 20. In the presently preferred embodiment, microcontroller 40 is an Atmel AT89C2051 microcontroller commercially available from Atmel Corporation, San Jose, Calif. The AT89C2051 is a low-voltage, high-performance CMOS 8-bit microcomputer with 2 K bytes of Flash programmable and erasable read only memory (PEROM).

Microcontroller 40 regulates operation of the electronics of device 10 through a plurality of different operating modes, each mode intended to treat via a specific variable waveform. In the preferred embodiment, microcontroller 40 allows for eight levels of therapeutic intensity.

As previously described, operating keys 16 are arranged in such a way that one can easily and quickly change any setting suitable to one's tolerance to the therapeutic voltage.

With continued reference to FIG. 2, the other main electronic components of device 10 include a pulse transformer 42 for generating the therapeutic pulses to be delivered through electrodes 20, a power supply 44 and voltage multiplier 46 for providing power to the pulse transformer 42 and microcontroller 40, a crystal oscillator 48 for providing a system clock signal to microcontroller 40, and a piezoelectric oscillator 50 for providing auditory cues relative to operation of device 10 to the user.

In operation, function keys 16 are used to control microcontroller 40 to operate in a desired therapy mode, as will be hereinafter described in further detail, which in turn is reflected in illumination of selected ones of LED indicators 18. Depending upon the selected mode, microcontroller 40 generates the appropriate waveform on an output line 50 to pulse transformer 42. Pulse transformer 42, in turn, amplifies the waveform into a higher voltage therapeutic pulse which is then applied to patient tissue via electrodes 20.

In addition to serving as the means for delivery of the electrotherapy, electrodes 20 are also preferably utilized to sense electrical conditions in the area of treatment, so as to provide bio-feedback to microcontroller 40 via lines 54 and 56 (through a Schmitt trigger circuit 58).

As previously noted, an alphanumeric display, for example, and LCD display or the like, can be provided in addition to or instead of LED indicators 18. Further, although not shown in FIG. 2, an interface such as a USB port can be provided to microcontroller 40 providing the ability to program, monitor, and diagnose operation of device 10 using an external control system such as a computer.

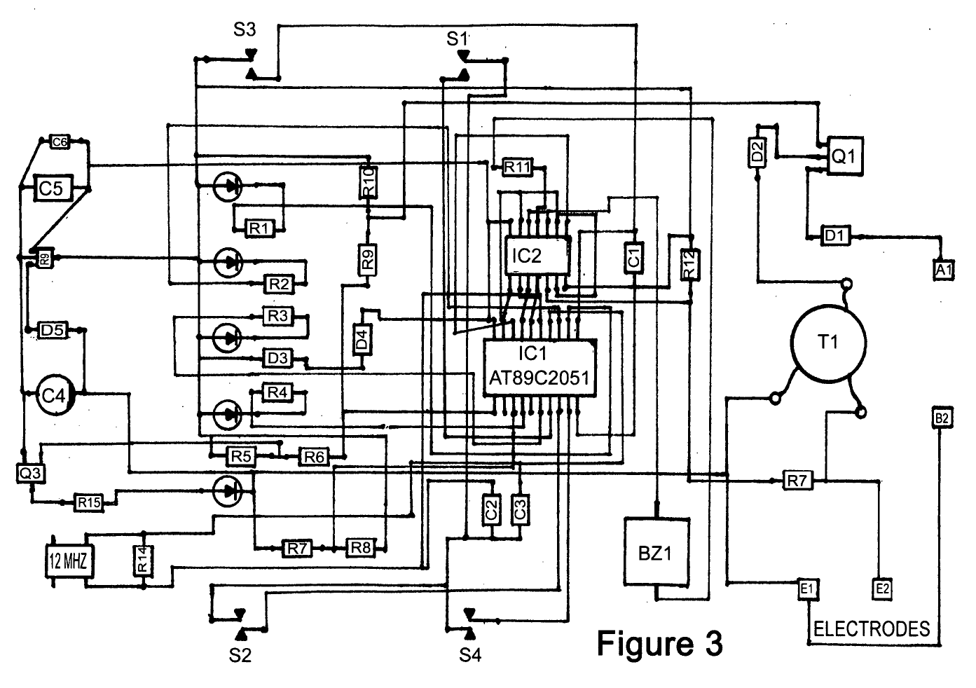

FIG. 3 is a schematic diagram of the presently disclosed embodiment of the invention. The circuit components shown in the schematic of FIG. 3 are specified in the following Table 1:

TABLE 1

| COMPONENT | DESCRIPTION |
|---|---|
| IC1 | Atmel AT89C2051-24 8-bit microcontroller |
| IC2 | ST Microelectronics quad 2-input Schmitt triggers |
| Q1 | Motorola MJ032C transistor |
| Q2 | Infineon BAW79D transitor |
| Q3 | Phillips BC807-40 transistor |
| X1 | ECS 24 MHz crystal oscillator |
| C1 | 0.2 μF capacitor |
| C2 | 47 μF capacitor |
| C3 | 33 μF capacitor |
| C4 | 22 pF capacitor |
| C5 | 0.2 μF capacitor |
| C6 | 0.2 μF capacitor |
| R1 | 2 kΩ resistor |
| R2 | 2 kΩ resistor |
| R3 | 2 kΩ resistor |
| R4 | 2 kΩ resistor |
| R5 | 10 Ω resistor |
| R6 | 10 Ω resistor |
| R7 | 1.6 MΩ resistor |
| R8 | 1.6 MΩ resistor |
| R9 | 750 kΩ resistor |
| R10 | 1 kΩ resistor |
| R11 | 240 Ω resistor |
| R12 | 3 kΩ resistor |
| R13 | 3 kΩ resistor |
| R14 | 510 kΩ resistor |
| R15 | 2.2 MΩ resistor |
| R16 | 100 Ω resistor |
| BZ1 | Piezoelectric transducer |
| BH1 | 9 V batter holder |
| T1 | Pulse transformer |
| J1 | DC power jack |
| B1 | 9 V battery |

Of course, it is to be understood that the present invention is not limited to the specific implementation shown in FIG. 3, and it is believed that those of ordinary skill in the art having the benefit of the present disclosure could readily implement an embodiment of the invention having the functionality of the disclosed embodiment in alternative ways.

Unlike other prior art therapeutic stimulation systems discussed above, the presently disclosed embodiment of the invention introduces a variable, high frequency electronic wave form through the skin and into the body of a person. As will be hereinafter described in further detail, and in accordance with one aspect of the invention, device 10 may receive bio-feedback from the patient, in order that it can vary the therapy being administered according to changes sensed during application of the therapy.

The wave forms are introduced through epidermal contact of the electrode head of the apparatus with patient tissue. In accordance with a significant aspect of the invention, and unlike prior art systems, the present invention does not utilize wires to make contact between a pulse generator and the patient. The present invention is a handheld, wireless device adapted to be manipulated by a user over injured tissue in order to effectively treat the patient. The electrode head is configured so that the electrical conduction from one of two electrodes (or in an alternative configuration three electrodes), that are separated from each other by a distance of four (or as many as eight) millimeters apart, introduces a current flow between said electrodes through the surface of the integument. The surface flow is considered to be micro-transcutaneous, and is used by the apparatus herein disclosed to measure changes of the electrical properties of the integument during treatment of a person.

In the disclosed embodiment, the high frequency electronic wave forms are introduced into a person's body in a series or stream of modulated (on-off) pulses. In accordance with one aspect of the invention, the frequency, modulation (described below), amplitude, damping, rectification, polarity, and duration of pulses 60 are variable parameters which may be manually or automatically selected by a user.

In one embodiment, the therapeutic regimen delivered by device 10 consists of a continuous burst of pulses for a first predetermined interval, followed by a pause for a second predetermined interval, after which another continuous burst commences. For the purposes of the present disclosure, the following nomenclature is used to describe the modality of the therapeutic regiment: <X:Y>, where X is the duration, in seconds, during which pulses 60 are continuously delivered, and Y is the duration, in seconds, of the pause following each stream of pulses 60.

For example, <1:1> modulation means one second of pulses 60 followed by one second of pause, followed by another one second of pulses 60, and so on. In the presently disclosed embodiment, the most powerful setting is <5:1> and the least powerful setting is <1:5>.

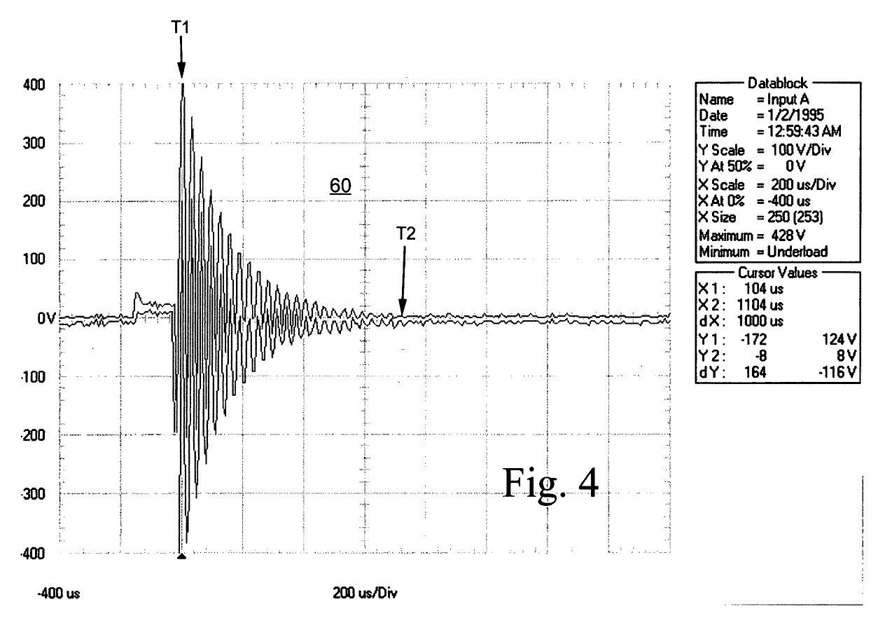

FIG. 4 shows an example of an unrectified, damped sinusoidal pulse 60 in accordance with one embodiment of the invention. Preferably, device 10 is programmable, either manually or automatically, to permit the selection of, among other features, the damping constant of damped pulses such as pulse 60 in FIG. 4. That is, the rate at which the pulse decreases from its initial, maximum amplitude, such as at time T1 in FIG. 4, to its minimum amplitude (i.e., 0 V), may be selectively programmed. In the presently disclosed embodiment, the amplitude of therapeutic pulses may range from 100 V to 1600 V, with a maximum current of approximately 100 mA. Additionally, the oscillating frequency of the pulses is selectable.

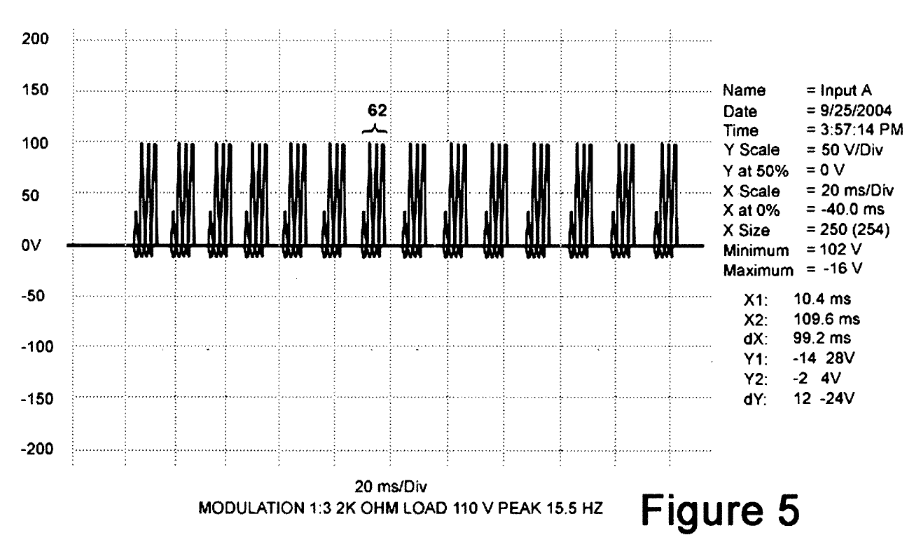

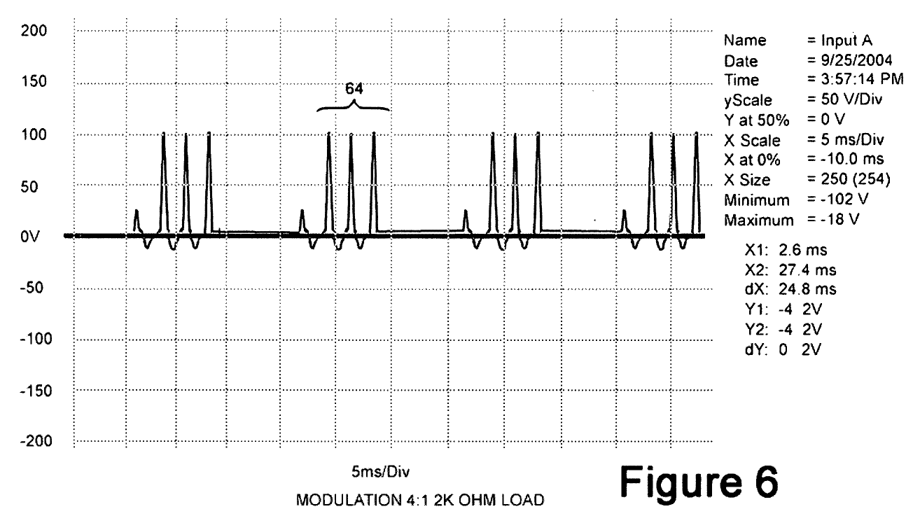

FIG. 5 shows an example of a <1:3> modulated therapy regimen consisting of a stream of rectified, undamped pulses, an exemplary burst of which being designated with reference numeral 62 in FIG. 5. FIG. 6 shows an example of a <4:1> modulated therapy regimen of rectified, undamped pulses, an exemplary burst of which being designated with reference numeral 64 in FIG. 6. FIGS. 5 and 6 also show that the polarity of the pulses can be selected, with those in FIGS. 5 and 6 being limited to positive voltages.

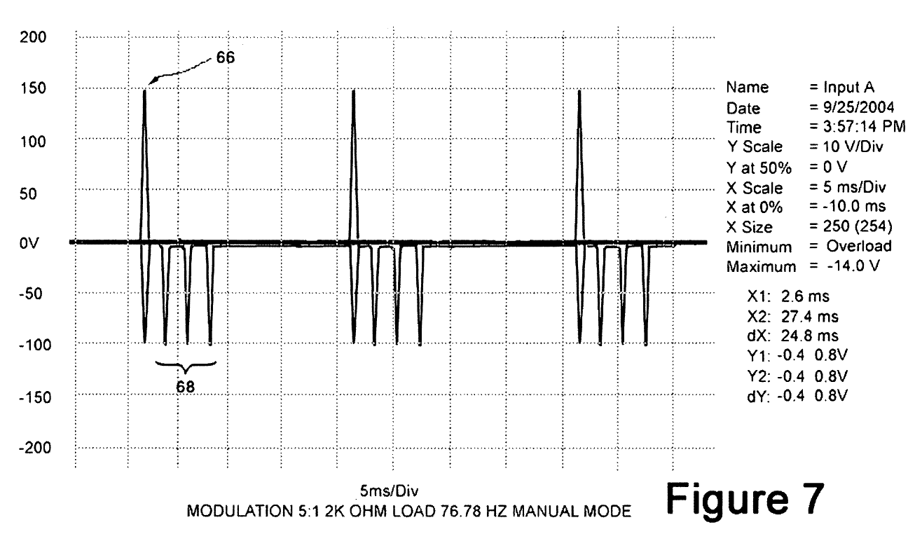

FIG. 7 shows an example of a <5:1> modulated therapy regimen, with a hybrid polarity setting consisting of an initial pulse 66 between a maximum positive and maximum negative polarity, followed by a burst of undamped, negative-polarity pulses 68.

In accordance with one aspect of the invention, device 10 is intended to obtain three types of effects on a patient: stimulation, harmonization, and sedation. Stimulating refers to reinforcing the area being treated, such that the tissue energy level is increased. Harmonizing refers to enhancing a relatively normal state in the patient, usually at the conclusion of a treatment session. Sedation refers to the treatment of an acute or inflammatory condition, where the objective is to lower or decrease the energetic activity in the area being treated.

In the presently preferred embodiment, stimulation therapy involves a therapeutic regimen within the following operational parameters:

Stimulation
Amplitude: Low
Frequency: Low, e.g., less than 60 Hz
Modulation: <1:3> to <1:5>
Damping: Off

Harmonizing
Amplitude: Medium
Frequency: 30-120 Hz
Modulation: <1:1>
Damping: Selectable

Sedation
Amplitude: High
Frequency: High, e.g., greater than 120 Hz
Modulation: <3:1> through <5:1>
Damping: Selectable

The electrical properties of the cell are a function of the energy state of the individual cells, specifically, the concentration of the adenosine triphosphate (ATP) that is present in the peripheral cytoskeleton of the cells. Too much energy is typically associated with an inflammatory process; while too little energy is typically associated with the onset (or progression) of degenerative disease processes.

When operating device 10, the handle is gripped generally in the area designated with reference numeral 14, the power switch 16-1 is turned on, and electrodes 20 are applied to the skin and is moved back and forth. When in operation, treatment is guided by a visual display of lights 18 on the back of housing 12. Treatment is also guided by observation of the responses of the treated area. These responses include: reddening of the skin, numbness, and, in accordance with one aspect of the invention, tactile feedback in the form of a sensation of “stickiness,” i.e., resistance to movement of the device across the area being treated, as the device is drawn across the patient's skin (i.e., the device will give the sensation of being magnetically dragged), and audible feedback in the form of an increase in the electronic chirping-buzzing sound made by the device. The sound is generated by changes in the electrical flux. These sensed changes activate a circuit that in turn activates a piezoelectric crystal to produce a range of sounds.

The tactile and audible feedback generated by the piezoelectric crystal are an active part of any treatment process, and reflect the progress of the electrical stimulation in the course of operating the apparatus. The sounds serve to guide the progress of electrical stimulation. In the movement of the device over the surface of the skin, there is information generated to guide the operator, and this information is in the form of an audible sound or buzzing that is related to the changes sensed in the electrical properties of the skin being treated. The tactile feedback likewise serves as an indication of the progress of the therapy.

The variable, high frequency electronic wave forms comprise a high-voltage stimulation that penetrates through the layers of the integument and causes electrical displacement current effects through the dielectric polarization of the three layers of: (1) the integument; (2) the highly conductive fascial tissue that is just under the integument that encloses the muscle tissue underneath; and (3) the musculature. The fascial layer is electrically charged in the course of the therapeutic stimulation.

While the stimulation is only transcutaneous at the surface area in contact with the electrode head, the displacement current causes the variable high frequency electronic wave form stimulation to be propagated beyond the area of immediate contact of the electrode head into surrounding areas. The high voltage (and very low amperage) of the variable high frequency electronic wave form assures deep penetration of the areas being stimulated.

During treatment, there is an oscillation or change of electric flux through the skin (as in a capacitor) in relation to time. When one side of a capacitor is charged an electric impulse is propagated to the other side, even though there is no actual flow of electrons. The skin acts as a capacitor and the electrical stimulation propagates the output waveform to areas well beyond the immediate area of treatment, thus greatly enhancing its effectiveness.

As noted above, device 10 may be implemented to be selectively operable in one of two modes, characterized generally as a “manual” mode and an “automatic mode.” In manual mode, the user manually selects a mode, specifying values for the various programmable parameters discussed above, and applies continuous treatment according to the selected mode.

In automatic mode, device 10 has the ability to automatically measure of the rate-of-change in the electrical properties of the epidermis (as measured between electrodes 20), and this provides information for the processing of dynamic interactive regulation of the electrical stimulation. That is, the stimulation parameters may be automatically adjusted through a neuro-adaptive cybernetic program that is able to generate a real-time interactive process that is able to coax tissues into a balanced and coherent bioelectric state that is essential for healing. Whereas prior art TENS devices simply blast the patient with electrical stimulation, the apparatus in accordance with the presently disclosed embodiment can provide a bio-electrically balanced and effective treatment. The neuro-adaptive program can provide for periodic adjustment of one or more of the operational parameters discussed hereinabove to achieve optimal therapeutic effectiveness.

In automatic mode, in the pause intervals between pulses the electrical properties of the micro-transcutaneous electrical connection between electrode conductors may be sensed as the rate-of-change of electrical impedance. Electrical changes thus sensed provide information to processor 40. Processor 40 is programmed to analyze incoming information and utilizes an algorithm to make modifications to the wave form of subsequent pulses in the series or stream of pulses. Accordingly, the variable high frequency electronic waveforms that are transdermally introduced into the body of a person are regulated through a dynamic cybernetic adaptive regulation process so that the body receives the most beneficial stimulation possible.

The micro-transcutaneous method of stimulating nerve tissue (particularly the C-fiber neurons in the peripheral nervous system) through a single moving electrode head is found to be more effective in relieving pain than the presently established medical practice called transcutaneous electrical nerve stimulation (TENS). In operation, the apparatus is “wireless” because the metal contacts of the electrode head make direct contact with the skin. No TENS electrode pads and connecting wires are ever needed.

The apparatus is biologically most active with neuronal C-fibers, which comprise 85% of all nerves in the body, and these fibers react most readily to the stimulation. This stimulation enhances the output of cellular energy (adenosine triphosphate or ATP), and the production of neuropeptides (and precursor peptides), and improves their transport throughout the length of the nerve fibers.

When the body has a normal flow of energy and information it quickly heals from injury, disease or toxicity. However, when this flow is blocked, the body can become accustomed to an imbalanced or pathological state. The present invention is able to stimulate cellular biological processes to speed the restoration of normal function. This opens up the normal flow of energy and information, and healing moves quickly to completion.

The neurons under treatment may be low-functioning and unable to produce enough ATP and neuropeptides to re-establish the body's natural healthy state. Thus, the treatment protocols are designed to restore healthy levels of ATP and neuropeptide production. Neurons may also have too much ATP and inflammatory neuropeptides (e.g. histamine), and the adaptive ability of the aforementioned apparatus treatment provides appropriate stimulation to restore bio-electrical balance (essential for bio-chemical balance).

Following treatment, the increased levels of ATP and neuropeptides last up to several hours; thus, the healing process will continue long after the treatment is over. Thus, treatment on one area can benefit the chemical imbalances in another different area or often the whole body. Thus, there is often some collateral benefit of localized treatment that may correct insomnia, digestive problems, depression and other emotional problems.

Although specific embodiments and variations of the invention have been disclosed herein in some detail, this has been done solely for the purposes of describing various features and aspects of the invention, and is not intended to be limiting with respect to the scope of the invention. It is contemplated that various substitutions, alterations, and/or modifications, including but not limited to those implementation variations which may have been suggested in the present disclosure, may be made to the disclosed embodiments without departing from the spirit and scope of the invention as defined by the appended claims, which follow.

What is claimed is:

1. A wireless, handheld electrotherapeutic device, comprising:
an external housing adapted to be grasped generally at a base portion thereof by a user's hand;
a plurality of electrodes disposed on a distal surface of said housing;
internal pulse generating circuitry adapted to generate a stream of electrical pulses between said plurality of electrodes;
wherein said distal surface is adapted to be brought into contact with a patient's skin thereby electrically stimulating said patient's neural tissue in a localized area.

2. A wireless, handheld electrotherapeutic device in accordance with claim 1, wherein said stream of electrical pulses comprises a repeating pattern of a burst of pulses generated for a first selected time interval followed by a pause for a second selected time interval.

3. A wireless, handheld electrotherapeutic device in accordance with claim 2, wherein said bust of pulses comprises a succession of damped sinusoidal pulses.

4. A wireless, handheld electrotherapeutic device in accordance with claim 2, wherein said the duration of said first and second intervals are programmable by a user.

5. A wireless, handheld electrotherapeutic device in accordance with claim 3, wherein the damping constant of said damped sinusoidal pulses is programmable.

6. A wireless, handheld electrotherapeutic device in accordance with claim 4, wherein the amplitude of said electrical pulses is programmable.

7. A wireless, handheld electrotherapeutic device in accordance with claim 6, wherein the polarity of said electrical pulses is programmable.

8. A wireless, handheld electrotherapeutic device in accordance with claim 7, wherein the oscillating frequency of said electrical pulses is programmable.

9. A wireless, handheld electrotherapeutic device in accordance with claim 2, wherein said device further comprises an oscillator for providing audible feedback reflecting progress of electrotherapeutic treatment of said localized area.

10. A wireless, handheld electrotherapeutic device in accordance with claim 2, wherein said device provides tactile feedback reflecting progress of electrotherapeutic treatment of said localized area.

11. A wireless, handheld electrotherapeutic device in accordance with claim 10, wherein said tactile feedback comprises resistance against movement of said electrodes over said localized area.

12. A wireless, handheld electrotherapeutic device in accordance with claim 2, wherein a plurality of operational parameters of said device are programmable.

13. A wireless, handheld electrotherapeutic device in accordance with claim 12, wherein said programmable operational parameters are programmed by a user.

14. A wireless, handheld electrotherapeutic device in accordance with claim 12, wherein said programmable operation parameters are automatically programmed during an electrotherapeutic treatment session.

15. A wireless, handheld electrotherapeutic device in accordance with claim 14, further comprising:
internal sensing circuitry, coupled to at least one of said electrodes, for sensing electrical conditions at said localized area.

16. A wireless, handheld electrotherapeutic device in accordance with claim 15, wherein said internal sensing circuitry senses electrical impedance at said localized area.

17. A wireless, handheld electrotherapeutic device in accordance with claim 16, wherein said internal sensing circuitry senses electrical impedance at said localized area during said pause of said second predetermined time interval.

18. A wireless, handheld electrotherapeutic device in accordance with claim 16, wherein said sensing circuitry and said pulse generating circuitry are cooperatively responsive to changes in sensed electrical impedance to modify at least one of said programmable operational parameters of said device.

19. A method of electrotherapeutic treatment of a patient, comprising:
providing a wireless, handheld electrotherapeutic device having a plurality of electrodes disposed on a distal surface thereof;
controlling said device to generate electrical stimulating pulses between said electrodes; and
bringing said electrodes into contact with said patient at a localized area to be treated, such that said electrical stimulating pulses are delivered to said patient.

20. A method in accordance with claim 19, wherein said step of controlling said device to generate electrical stimulating pulses comprises controlling said device to deliver a repeating pattern of a burst of pulses generated for a first selected time interval followed by a pause for a second selected time interval.

21. A method in accordance with claim 20, wherein said bust of pulses comprises a succession of damped sinusoidal pulses.

22. A method in accordance with claim 20, further comprising selecting the durations of said first and second time intervals.

23. A method in accordance with claim 21, further comprising selecting the damping constant of said damped sinusoidal pulses.

24. A method in accordance with claim 20, further comprising selecting the maximum amplitude of said electrical pulses.

25. A method in accordance with claim 24, further comprising selecting the polarity of said electrical pulses.

26. A method in accordance with claim 25, further comprising selecting the oscillating frequency of said electrical pulses.

27. A method in accordance with claim 20, further comprises providing an oscillator for providing audible feedback reflecting progress of electrotherapeutic treatment of said localized area.

28. A method in accordance with claim 27, further comprising adjusting at least one operational parameter of said device in response to said audible feedback during a treatment session.

29. A method in accordance with claim 20, further comprising adjusting at least one operational parameter of said device in response to tactile feedback reflecting progress of electrotherapeutic treatment of said localized area.

30. A method in accordance with claim 29, wherein said tactile feedback comprises resistance against movement of said electrodes over said localized area.

31. A method in accordance with claim 20, further comprising programming a plurality of operational parameters of said device.

32. A method in accordance with claim 31, wherein said step of programming said plurality of programmable operation parameters occurs automatically during an electrotherapeutic treatment session.

33. A method in accordance with claim 32, further comprising sensing electrical conditions at said localized area.

34. A method in accordance with claim 33, wherein said step of sensing electrical conditions at said localized area comprises sensing electrical impedance at said localized area.

35. A method in accordance with claim 34, wherein said step of sensing electrical conditions at said localized area occurs during said second selected time interval.

36. A method in accordance with claim 35, further comprising adjusting at least one operational parameter of said device in response to changes in sensed electrical impedance.
